(19) United States
(12) Patent Application Publication
(10) Pub. No.: US 2025/0235704 A1
(43) Pub. Date: Jul. 24, 2025

(54) Active Medical Device Having A Dual Band Antenna Configuration Inside A Non-Conductive Enclosure Connected To A Metallic Sleeve Housing An Electrochemical Cell

(71) Applicant: Greatbatch Ltd., Clarence, NY (US)

(72) Inventors: Gabriel Amilcar Barbat, Ciudad de la Costa (UY); Camila Maria Duarte, Montevideo (UY); Rocio Belen Curiel, Montevideo (UY)

(21) Appl. No.: 19/032,596

(22) Filed: Jan. 21, 2025

Related U.S. Application Data
(60) Provisional application No. 63/622,212, filed on Jan. 18, 2024.

(51) Int. Cl. A61N 1/372; A61N 1/375; A61N 1/378

(52) U.S. Cl. CPC A61N 1/37229; A61N 1/3754; A61N 1/3758; A61N 1/3787

(57) ABSTRACT

An active medical device (AMD) has a low-profile ceramic housing that contains a communication antenna/inductive charging coil assembly that is hingedly connected to a PCB assembly. The communication antenna/inductive charging coil assembly is folded over the PCB assembly to provide a compact communication antenna/inductive charging coil assembly/PCB assembly that fits inside the ceramic housing. The ceramic housing does not interfere with communication/charging fields that are transmitted between external devices and the AMD when it is implanted in body tissue or worn outside the body. The PCB assembly draws power from a power source housed in a titanium sleeve connected to a distal end of the ceramic housing. A lead is connected to a feedthrough connector assembly at a proximal end of the ceramic housing. Electrodes of the lead are energized by the PCB assembly to provide electrical stimulation to body tissue, sense biological signals from body tissue, or both.

### CROSS-REFERENCE TO RELATED APPLICATION

This application claims priority to U.S. provisional application Ser. No. 63/622,212, filed on Jan. 18, 2024.

### BACKGROUND OF THE INVENTION

### 1. Field of the Invention

The present invention relates to the fields of implantable and externally worn medical devices. More particularly, the present invention relates to an active medical device (AMD) that is detachably connected to a lead. The lead is preferably an implantable lead that is designed to send electrical stimulation pulses to the surrounding body tissue of a patient, sense biological signals from the body tissue, or both stimulate and sense.

The AMD of the present invention has a power source connected to the distal end of a printed circuit board (PCB) assembly for powering its electronic components. The electronic components control the various functions of the medical device including, but not limited to, electrical stimulation of body tissue and biological sensing. A lateral edge of the PCB is connected to a communication antenna/inductive charging coil assembly that provides for remote two-way communication with an external device and inductive wireless charging of the power source. Having the communication antenna/inductive charging coil assembly connected to a lateral edge of the PCB means that the subassembly can be folded over the PCB to provide a compact PCB assembly that fits inside a low-profile ceramic housing. The power source connected to the distal end of the PCB assembly also has a low-profile. A proximal end of the ceramic housing for the PCB assembly is connected to a feedthrough connector assembly which, in turn, is connected to a strain-relief device for a lead, preferably an implantable lead. Since the communication antenna/inductive charging coil assembly is a lateral extension of the PCB, the AMD has a relatively thin or low-profile with a reduced total volume for less invasive therapies. Preferably, an AMD according to the present invention has a volume of less than 3 cc. including its power source.

### 2. Prior Art

It is typical for an AMD that is powered by a rechargeable power source to support its communication antenna/inductive charging coil assembly in an epoxy header connected to the device housing or to support the subassembly on an external surface of the housing. This means that the communication antenna/inductive charging coil assembly supported in the epoxy header is typically spaced from the PCB contained inside the device housing, which requires electrical connections from the PCB to the subassembly. In addition to the epoxy header, these electrical connections add bulk to the AMD, which can be significant. Otherwise, mounting the communication antenna/inductive charging coil assembly on an outer surface of the device housing also increases the thickness of the AMD, which is added bulk.

Moreover, when the communication antenna/inductive charging coil assembly is mounted on the device housing, the metallic material of the housing behind the assembly can cause eddy currents which can negatively affect remote inductive or RF charging signals and severely limit communication between the medical device and a remote programmer or interrogation device. Remote programmers are used by the patient and the clinician to configure the medical device to operate in a desired manner. A patient programmer is used by the patient in whom the medical device is implanted to adjust the parameters of electrical stimulation delivered by the device. A clinician programmer is used by medical personnel to configure and adjust stimulation parameters that the patient is not permitted to control.

Therefore, there is a desire to minimize the total volume of an AMD so that the medical device is as small as possible. According to the present invention, a considerable reduction in volume is realized by connecting the communication antenna/inductive charging coil assembly to a lateral edge of the PCB and then folding the assembly over the PCB. The resulting relatively thin or low-profile PCB assembly is contained inside a ceramic housing of the medical device. A distal end of the ceramic housing is connected to a metallic sleeve that fits over the casing for a power source powering the electronic components of the PCB. A proximal end of the ceramic housing is connected to a feedthrough connector assembly which is detachably connected to a strain relief device for a lead, preferably an implantable lead. The resulting AMD is significantly smaller than an AMD having a metallic housing, which is beneficial for patient safety and comfort. Preferably, the AMD of the present invention has a volume of less than 3 cc. including its power source.

### SUMMARY OF THE INVENTION

The purpose of the present invention is to reduce the size of an active medical device (AMD), preferably an active implantable medical device (AIMD), by reducing the volume that is occupied by the communication antenna/inductive charging coil assembly connected to a printed circuit board (PCB). Unlike the conventional constructions describes in the Prior Art section above, the communication antenna/inductive charging coil assembly, whether there are two dedicated antennas/coils or an integrated antenna, are contained inside a ceramic housing of the AMD. Alumina is a preferred ceramic as it is an inherently electrically insulative material and readily permits two-way communication and inductive charging without creating detrimental eddy currents. Since both the traditional epoxy header and the alternative method of mounting the communication antenna/inductive charging coil assembly on an outer surface of the device housing are eliminated with the present AMD, a medical device of a significantly reduced size is the result. A smaller device is easier to implant in a patient and would be expected to cause less trauma to the patient. A smaller medical device would also be expected to be less cumbersome when worn on a patient's body.

In that respect, the AMD of the present invention has a non-conductive housing that is made from a ceramic material, for example, alumina, which is an inherently electrically insulative material. The ceramic housing contains a PCB assembly hingedly connected to a PCB and a communication antenna/inductive charging coil assembly. The PCB has electronic circuits that draw power from an electrical power source connected to a distal end of the ceramic housing. A lead, preferably an implantable lead, is detachably connected to a feedthrough connector assembly at a proximal end of the ceramic housing. In that manner, electrodes of the implantable lead are energized by the PCB assembly to either provide electrical stimulation to body tissue in which the medical device is implanted, sense biological signals from adjacent body tissue, or both.

The hingedly connected aspect of the present invention means that the communication antenna/inductive charging coil assembly is folded over or moved into a position that is aligned directly spaced above or directly spaced below the PCB assembly. In its folded and aligned position, the communication antenna/inductive charging coil assembly is aligned directly spaced above or directly spaced below the PCB assembly. The communication antenna/inductive charging coil assembly folded over the PCB assembly is then fit into a low-profile ceramic housing of the AMD. Importantly, the ceramic housing does not interfere with communication/charging fields that are transmitted between external devices and the AMD when it is implanted in body tissue or worn outside the body. These include communication signals from patient and clinician programmers to the medical device, and inductive or RF (radio frequency) charging signals sent from an external charging transmitter to a charging antenna connected to the power source. This means that the communication and charging antennas can be hingedly connected to the PCB assembly and folded over the PCB assembly and then fitted inside the ceramic housing, which further reduces the AMD's volume and manufacturing complexity.

These and other aspects of the present invention will become increasingly more apparent to those skilled in the art by reference to the following detailed description and to the appended drawings.

### BRIEF DESCRIPTION OF THE DRAWINGS

FIG. 1 is a wire formed diagram of a generic human body showing a number of medical devices 100A to 100L according to the present invention that can either be implanted in a patient's body tissue or attached externally to the body.

FIG. 2 is a simplified block diagram of an exemplary medical system 10 according to various embodiments of the present invention.

FIG. 3 is a perspective view of an exemplary active medical device (AMD) 12 that is detachably connected to an implantable lead 36.

FIG. 4 is an exploded view of the exemplary AMD 12 shown in FIG. 3.

FIG. 5 is a plan view showing a printed circuit board assembly 34 comprising electronic circuits 38 mounted to a printed circuit board (PCB) 36 and with a communication antenna/inductive charging coil assembly 52 hingedly connected to a lateral edge 42 of the PCB 36 by the living hinges 62, 64 and 66.

FIG. 5A is a plan view showing the printed circuit board assembly 34 of FIG. 5 connected to a proximal end 44 of the printed circuit board 36 of the PCB assembly 34.

FIG. 5B is a plan view showing the printed circuit board assembly 34 of FIG. 5 connected to a distal end 46 of the printed circuit board 36 of the PCB assembly 34.

FIG. 6 is a perspective, partly broken away, view of the exemplary AMD 12 of FIG. 3 with the PCB assembly 34 removed from its ceramic housing 26 and with the communication antenna/inductive charging coil assembly 52 being folded over the PCB assembly.

FIG. 7 is an enlarged plan view of the exemplary AMD 12 shown in FIG. 3.

### DETAILED DESCRIPTION OF THE PREFERRED EMBODIMENTS

As used herein, the term “active medical device” means a medical device, whether implantable or worn externally, that is designed to deliver electrical stimulation to a patient's body tissue or sense biological signals from body tissue, or both stimulate and sense. An AIMD is an active medical device that is intended to be implanted in body tissue.

Further, as used herein, the term PCB assembly defines a printed circuit board (PCB) as a laminated sandwich structure of conductive and insulating layers, each with a pattern of traces, planes and other features (similar to wires on a flat surface) etched from one or more sheet layers of copper laminated onto or between sheet layers of a non-conductive substrate. PCBs are used to connect or wire electronic components to one another in an electronic circuit. Electrical components may be fixed to conductive pads on the outer layers, generally by soldering, which both electrically connects and mechanically fastens the components to the board. PCB assemblies also typically include vias, which are metal-lined drilled holes that enable electrical interconnections between conductive layers with more than a single side. In the present invention, the PCB can be rigid, flexible or of a partially flexible, partially rigid design.

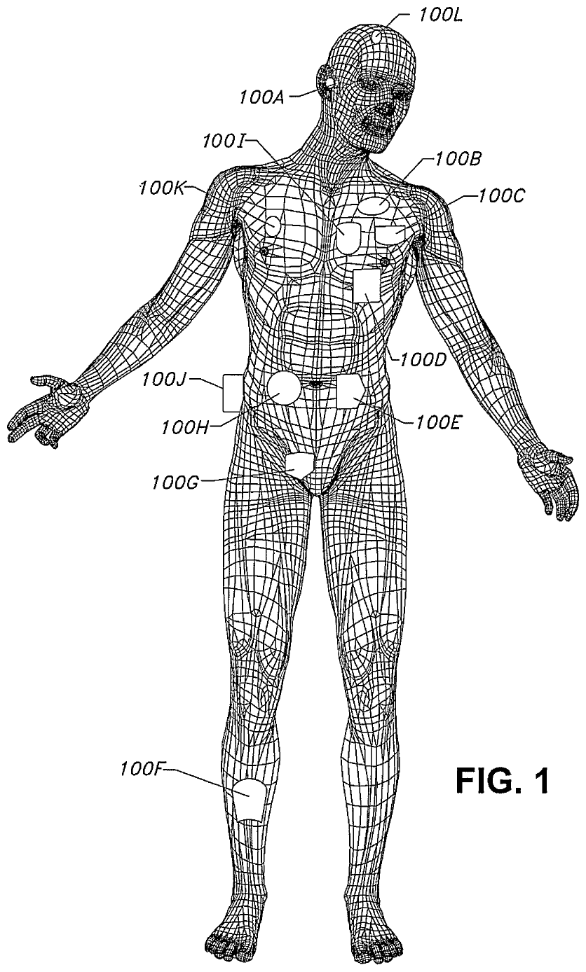

Turning now to the drawings, FIG. 1 is a wire form diagram of a generic human body illustrating various types of active implantable and external medical devices according to the present invention that can either be implanted in a patient's body or attached externally to the body.

Numerical designation 100A represents a family of hearing devices which can include the group of cochlear implants, piezoelectric sound bridge transducers, and the like.

Numerical designation 100B represents a variety of neurostimulators, brain stimulators, and sensors. Neurostimulators are used to stimulate the Vagus nerve, for example, to treat epilepsy, obesity, and depression. Brain stimulators are pacemaker-like devices and include electrodes implanted deep into the brain for sensing the onset of a seizure and also providing electrical stimulation to brain tissue to prevent a seizure from actually occurring. The lead wires associated with a deep brain stimulator are often placed using real time MRI imaging. Sensors include optical sensors, motion sensors, acoustic sensors, pressure sensors, analyte sensors, and electromagnetic sensors, among others.

Numerical designation 100C shows a cardiac pacemaker which is well-known in the art.

Numerical designation 100D includes the family of left ventricular assist devices (LVADs), and artificial heart devices.

Numerical designation 100E includes a family of drug pumps which can be used for dispensing insulin, chemotherapy drugs, pain medications, and the like.

Numerical designation 100F includes a variety of bone growth stimulators for rapid healing of fractures.

Numerical designation 100G includes urinary incontinence devices.

Numerical designation 100H includes the family of pain relief spinal cord stimulators and anti-tremor stimulators. Numerical designation 100H also includes an entire family of other types of neurostimulators used to block pain.

Numerical designation 100I includes a family of implantable cardioverter defibrillator (ICD) devices and also the family of congestive heart failure (CHF) devices. They are also known in the art as cardio-resynchronization therapy devices, otherwise known as CRT devices.

Numerical designation 100J illustrates an externally worn pack. This pack could be an external insulin pump, an external drug pump, an external neurostimulator or even a ventricular assist device.

Numerical designation 100K illustrates one of various types of EKG/ECG external skin electrodes which can be placed at various locations on the patient's body.

Numerical designation 100L represents external EEG electrodes that are placed on the patient's head.

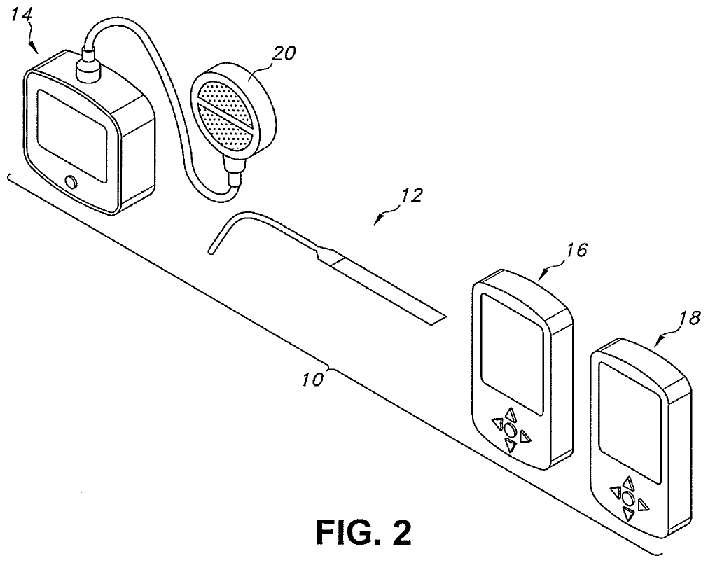

To provide context to the various medical devices 100A to 100L illustrated in FIG. 1, FIG. 2 illustrates a simplified block diagram of an exemplary medical device system 10 according to the present invention. The medical device system 10 includes an active medical device (AMD) 12, which is any one of various types of the medical devices that include a lead, whether implantable or external, as described above with reference to FIG. 1. The medical device system 10 also has an external charger 14, a patient programmer 16, and a clinician programmer 18.

The patient programmer 16 and the clinician programmer 18 may be portable handheld devices, such as a smartphone or other custom device, that are used to configure the AMD 12 so that the medical device can operate in a desired manner. The patient programmer 16 is used by the patient in whom the AMD 12 is implanted or worn externally. The patient may adjust the parameters of electrical stimulation delivered by the AMD 12, such as by selecting a stimulation program, changing the amplitude and frequency of the electrical stimulation, among other parameters, and by turning stimulation on and off. Additionally, the patient programmer 16 may collect and display data being collected by the medical device 12 and alert the patient to potential health risks.

The clinician programmer 18 is used by medical personnel to configure the other system components and to adjust stimulation parameters that the patient is not permitted to control. These include setting up stimulation programs among which the patient may choose and setting upper and lower limits for the patient's adjustments of amplitude, frequency, and other parameters. It is also understood that although FIG. 2 illustrates the patient programmer 16 and the clinician programmer 18 as two separate devices, they may be integrated into a single programmer in some embodiments.

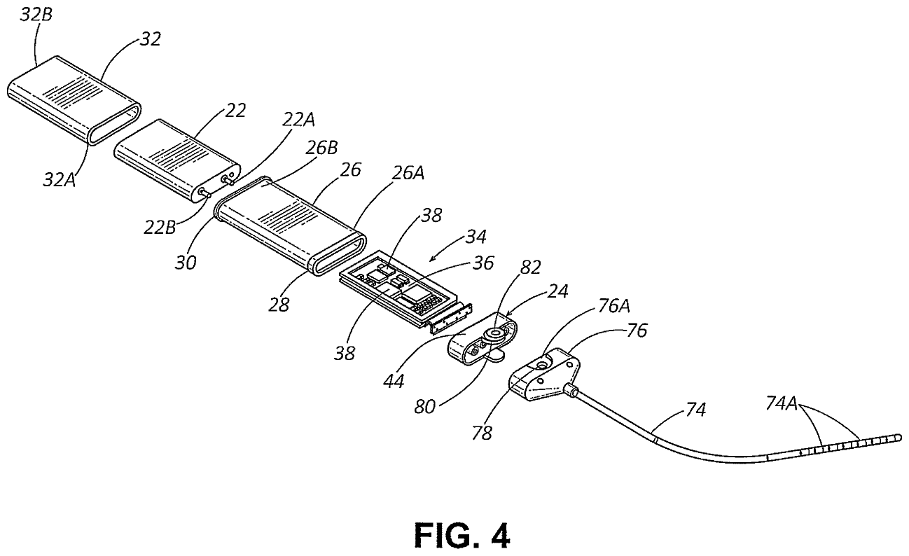

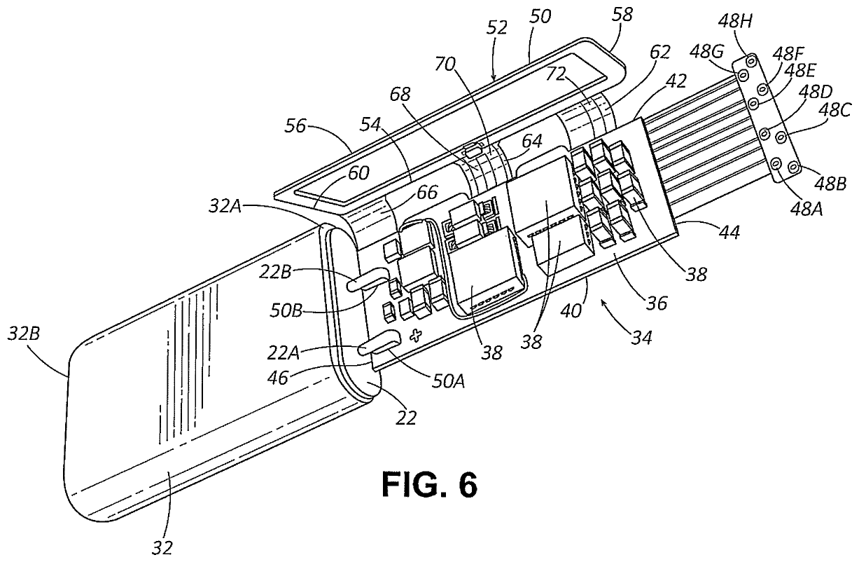

Electrical power can be delivered to the AMD 12 through an external charging pad 20 that is connected to the external charger 14. In some embodiments, the external charging pad 20 is configured to directly power the AMD 12 or it is configured to charge a rechargeable electrical power source 22 (FIGS. 4 and 6) of the medical device. The external charging pad 20 can be a hand-held device that is connected to the external charger 14, or it can be an internal component of the external charger. The external charger 14 and the charging pad 20 can also be integrated into a single device that is strapped on or attached to the patient with adhesive, and the like.

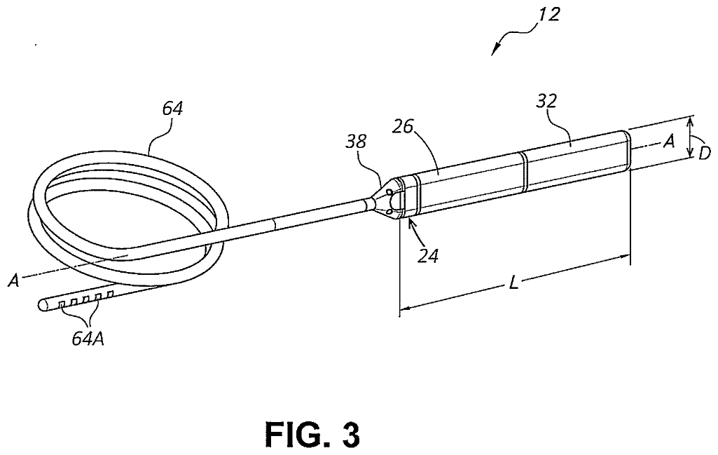

Referring now to FIG. 3, this drawing illustrates the AMD 12 as an exemplary embodiment of the various medical devices 100A to 100L illustrated in FIG. 1 and the exemplary AMD 12 shown in the medical device system 10 illustrated in FIG. 2 that can be implanted in a patient's body or worn externally on a patient's body. The AMD 12 is shown as an elongate device extending along a longitudinal axis A-A with an exemplary length “L” of about 47 mm and a width “W” of about 4 mm and comprising a feedthrough connector assembly 24 that is connected to the proximal end of a non-conductive housing 26. A metallic sleeve 32 which houses a power source (not shown in FIG. 3) is connected to the non-conductive housing 26 opposite the feedthrough connector assembly 24.

The non-conductive housing 26 is preferably formed from a ceramic material that has a relatively high resistivity. An essentially pure alumina is a preferred ceramic material. The term “essentially pure alumina” refers to an alumina ceramic having the chemical formula Al₂O₃. “Essentially pure” means that the post-sintered alumina is at least 96% alumina. In a preferred embodiment, the post-sintered alumina is at least 99% high purity alumina. Moreover, as long as they are biocompatible and offer the appropriate mechanical robustness, other suitable non-conductive materials for the housing 26 include glass (e.g., high purity fused silica or HPFS) or plastic (e.g., PEEK).

Prior to sintering, alumina comprising the non-conductive housing 26 may be a paste, a slurry, or a green-state ceramic that is injection molded, powder pressed, and the like, into the desired shape of the housing 26 as a single monolithic structure. The green-state alumina ceramic is very pliable due to the organic binders and solvents that have been temporarily added to the system. As an assembly, the green-state ceramic housing 26 is then subjected to a controlled co-firing or co-sintering process in ambient air that comprises a binder bakeout portion, a sintering portion, and a cool down portion.

As shown in FIG. 4, the non-conductive housing 26 has a surrounding sidewall extending from an annular proximal rim 26A to an annular distal rim 26B. The proximal and distal rims 26A, 26B surround and define an open interior inside the housing 26. Proximal and distal weld rings 28 and 30, preferably made of titanium, are brazed to the respective proximal and distal annular rims 26A and 26B.

With respect to the proximal end of the non-conductive housing 26, to hermetically connect the weld ring 28 to the proximal rim 26A, the outwardly facing edge of the rim 26A is provided with a metallization (not shown). A suitable metallization comprises two metallization layers, a first adhesion layer that is directly applied to the outwardly facing edge of the rim 26A, and a second, wetting layer, which is applied on top of the adhesion layer. In a preferred embodiment, the adhesion layer is titanium, and the wetting layer is either molybdenum or niobium.

The adhesion and wetting metallization layers may be applied to the annular proximal rim 26A by thin and thick film technologies, such as printing, painting, plating, and deposition processes. Metallization processes include screen printing, pad printing, brush coating, direct bonding, active metal brazing, magnetron sputtering, physical vapor deposition, ion implantation, electroplating, and electroless plating. In an alternate embodiment, both the adhesion and wetting metallization layers may be provided by a single metallization layer. In a similar manner, the annular distal rim 26B is provided with a metallization. For the sake of simplicity, the adhesion and wetting layers contacted to both the proximal and distal rims 26A, 26B are intentionally not shown.

A braze pre-form, for example a gold ring-shaped preform, (not shown) is seated on each of the metallized rims 26A, 26B. The proximal weld ring 28/proximal gold-preform/ceramic housing 26/distal gold-preform/distal weld ring 30 assembly is then subjected to a brazing process, as is well known by those skilled in the art related to brazing a ceramic material to a metallic flange. The brazing process melts the gold to thereby form a hermetic seal joining the weld rings 28, 30 to the non-conductive housing 26 at the respective proximal and distal rims 26A, 26B.

The power source 22 shown in FIGS. 4 and 6 for the AMD 12 can be a capacitor or a rechargeable battery, for example a hermetically sealed rechargeable Li-ion battery. In that respect, the power source 22 is not limited to any one chemistry or even a rechargeable chemistry and can be of an alkaline cell, a primary lithium cell, a rechargeable lithium-ion cell, a Ni/cadmium cell, a Ni/metal hydride cell, a supercapacitor, a thin film solid-state cell, and the like. Preferably, the power source 22 is a lithium-ion electrochemical cell comprising a carbon-based or Li₄Ti₅O₁₂-based anode and a lithium metal oxide-based cathode, such as of LiCoO₂ or lithium nickel manganese cobalt oxide (LiNiₐMn_{b}Co_{1-a-b}O₂). The power source 22 can also be a solid-state thin film electrochemical cell having a lithium anode, a metal-oxide based cathode and a solid electrolyte, such as an electrolyte of LiPON (LiₓPO_{y}N_{z}). Regardless its chemistry, the power source 22 is housed inside a distal metallic sleeve 32. Titanium is a preferred material for the casing of the power source 22 and the sleeve 32.

As particularly shown in FIGS. 3, 4, 6 and 7, the metallic sleeve 32 has an annular sidewall extending from an annular proximal rim 32A to a closed distal end 32B. The proximal rim 32A surrounds an open interior which is sized and shaped to house the power source 22. As will be described in greater detail hereinafter, the power source 22 has outwardly extending opposite polarity terminals 22A and 22B that are configured to be electrically connected to a PCB assembly 34 housed inside the non-conductive housing 26.

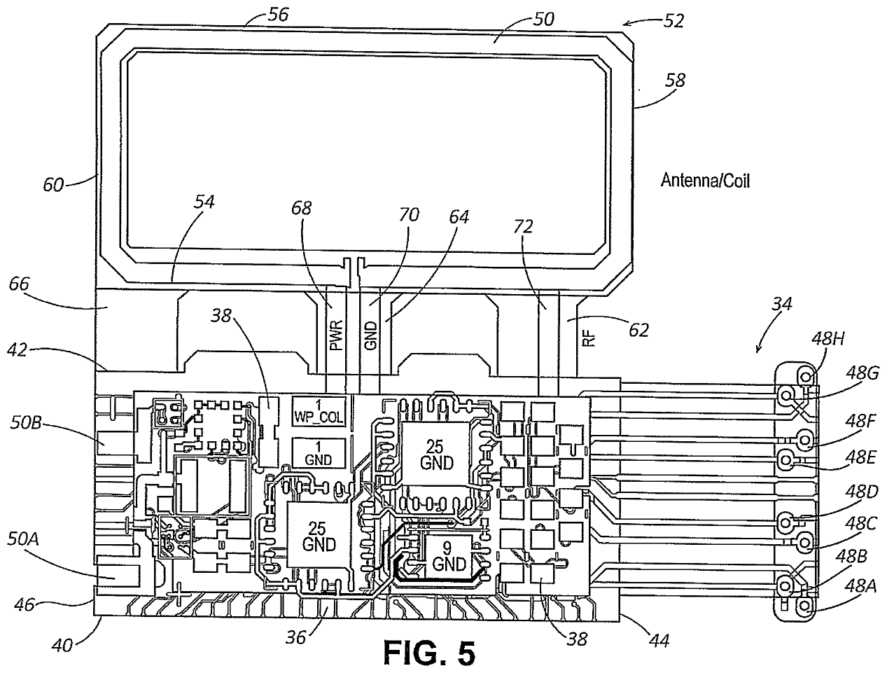

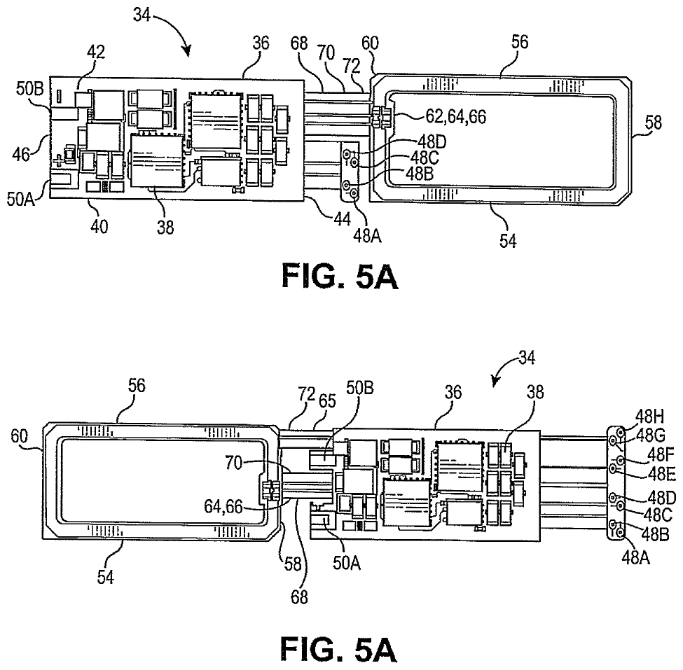

As shown in FIG. 5, the PCB assembly 34, which controls the various functions performed by the AMD 12, comprises a printed circuit board (PCB) 36 supporting at least one, and preferably a plurality of electronic components 38. The AMD functions include, but are not limited to, receiving sensed electrical signals pertaining to functions of the body tissue in which the AMD 12 is implanted and for delivering electrical current pulses to the body tissue.

The printed circuit board 36 has a length that comprises longitudinally extending right or upper and left or lower lateral edges 40 and 42 that meet with opposed proximal and distal ends 44 and 46. A plurality of electrical contacts, for example, 48A, 48B, 48C, 48D, 48E, 48F, 48G and 48H are supported on the PCB 36 at its proximal end or feedthrough connection end 44. While eight electrical contacts 48A to 48H are shown in FIG. 5, it is within the scope of the present invention that less than and possibly many more contacts can be supported on the printed circuit board 36. The number of electrical contacts is dictated by the type of AMD 12 and the medical condition it is designed to treat. Further, electrical contacts 50A and 50B are supported on the PCB 36 at its distal end or cell connection end 46. The proximal and distal electrical contacts 48A to 48H, 50A and 50B can be electrically conductive bond pads, solder bumps, circuit trace terminations, thru-hole pads, and the like.

In order to provide the AMD 12 as a device that can stay inside a patient's body for many years without needing to be replaced, a communication antenna/inductive charging coil assembly 52 is hingedly connected to the left lateral edge 42 of the PCB 36. The communication antenna/inductive charging coil assembly 52 has at least one to numerous winds of an electrically conductive wire or tape. The at least one, but preferably multiple turns of conductive wire or tape provide the communication antenna/inductive charging coil assembly 52 with a length that comprises longitudinally extending right or upper and left or lower coil lateral edges 54 and 56 that meet with opposed proximal and distal coil ends 58 and 60. The lengths of the lateral coil edges 54, 56 are substantially similar to the lengths of the lateral edges 40 and 42 of the PCB assembly 34. Similarly, the widths of the proximal and distal ends 58, 60 of the communication antenna/inductive charging coil assembly 52 are substantially similar to the widths of the opposed proximal and distal ends 44 and 46 of the PCB assembly 36.

After the distal weld ring 30 is hermetically connected to the distal rim 26B of the non-conductive housing 26 and with the power source 22 housed inside the metallic sleeve 32, the power source terminals 22A, 22B are connected to the contacts pads 50A, 50B of the PCB assembly 34. The proximal rim 32A of the sleeve 32 is then welded to the distal ring 30 of the housing 26. In this position, the power source terminals 22A and 22B connected to the electrical contacts 50A and 50B of the PCB assembly 34 provide electrical power to the printed circuit board 36 and its electronic components 38.

FIGS. 5 and 6 illustrate that the communication antenna/inductive charging coil assembly 52 is hingedly connected to the left lateral edge 42 of the PCB assembly 34 by three flexible extensions 62, 64 and 66 from the printed circuit board 36. Preferably, the flexible extensions 62, 64 and 66 are integral living hinges, which are thin flexible hinge made from the same material as the PCB 36 to which they are connected. If desired, the hinges 62, 64 and 66 are thinner than the thickness of the main PCB 36 to allow the communication antenna/inductive charging coil assembly 52 to bend along the line of the hinges with respect to the PCB assembly 34. Further, it is within the scope of the present invention that the assembly 52 can also be hingedly connected to the opposed right lateral edge 44.

In an alternate embodiment, FIG. 5A illustrates that the communication antenna/inductive charging coil assembly 52 is hingedly connected to the proximal end or feedthrough connection end 44 of the PCB assembly 34 by a flexible extension 63 from the printed circuit board 36. Preferably, the flexible extension 63 is an integral living hinge which is made from the same material as the PCB 36 to which it is connected. Desirably, the hinge 63 is thinner than the thickness of the main PCB 36 to allow the communication antenna/inductive charging coil assembly 52 to bend along the line of the hinge with respect to the PCB assembly 34.

In another alternate embodiment, FIG. 5B illustrates that the communication antenna/inductive charging coil assembly 52 is hingedly connected to the distal end or cell connection end 46 of the PCB assembly 34 by spaced-apart flexible extensions 65 and 67 from the printed circuit board 36. Preferably, the flexible extensions 65 and 67 are integral living hinges which are made from the same material as the PCB 36 to which they are connected. Desirably, the hinges 65 and 67 are thinner than the thickness of the main PCB 36 to allow the communication antenna/inductive charging coil assembly 52 to bend along the line of the hinges with respect to the PCB assembly 34.

In that respect, what is important is that in its folded configuration, whether connected to the right lateral edge 40, the left lateral edge 42, the proximal end 44 or the distal end 46, the communication antenna/inductive charging coil assembly 52 is positioned directly spaced above or directly spaced below the PCB assembly 36. Thus, it is an aspect of the present AMD 12 that the communication antenna/inductive charging coil assembly 52 is hingedly moved into a position that is aligned directly spaced above or directly spaced below the PCB assembly 34. In its folded and aligned configuration, the lateral edges 54 and 56 of the communication antenna/inductive charging coil assembly 52 are aligned directly spaced above or directly spaced below the respective lateral edges 42 and 40 of the PCB assembly 34, and the proximal and distal coil ends 58 and 60 are aligned directly spaced above or directly spaced below the respective proximal and distal ends 44 and 46 of the PCB assembly 34.

Moreover, while the communication antenna/inductive charging coil assembly 52 is shown in the drawings as a substantially rectangular structure, that is exemplary. In alternative embodiments, the communication antenna/inductive charging coil assembly 52 has an oval shape, a circular shape or a multi-sided shape that is neither rectangular or circular but has both straight sides and curved sides. Additionally, the communication antenna/inductive charging coil assembly 52 need not have a length and a width that substantially aligns with the length and width of the PCB assembly 34. In those alternate embodiments, the communication antenna/inductive charging coil assembly 52 has a length or width that is either greater or less than that of the PCB assembly 34.

The communication antenna/inductive charging coil assembly 52 is connected to the electronic circuits 38 supported on the PCB 36 by a power trace 68, a ground trace 70 and an RF signal trace 72. There is one or more capacitors on the communication antenna/inductive charging coil assembly 52 close to line 68 for resonating with the coil. These capacitors are placed directly on the coil in order to reduce power losses on lines 68 and 70. Trace 72 is configured to carry an RF signal from the communication antenna/inductive charging coil assembly 52 to a dedicated circuit or circuits on the PCB assembly 36 that are configured to convert RF or inductive energy signals received from the external charging pad 20 connected to the external charger 14 (FIG. 2) into a direct current voltage to charge the power source 22 to power the electronic components 38 of the PCB assembly 34. The communication antenna/inductive charging coil assembly 52 is also configured to receive communication signals from the patient and clinician programmers 16, 18 and send communication signals from the medical device 12 back to the programmers 16 and 18 related to electrical stimulation of body tissue, sensed biological signals from body tissue, or both stimulate and sense RF signals.

In that respect, the communication antenna/inductive charging coil assembly 52 may be used to receive both charging signals (e.g., inductive energy in the 13.56 MHz ISM band) and telemetry signals (e.g., Bluetooth LE or BLE). Thus, the PCB assembly 34 includes electronic components that allow charging signals in the 13.56 MHz ISM band to pass through but will reject signals outside the 13.56 MHz ISM band while charging the electrical power source 22, including telemetry signals in the 2.45 GHz ISM band (BLE). Moreover, the electronic components 38 of the PCB assembly 34 allow telemetry signals to pass when the AMD 12 is communicating with an external device, such as the patient and clinician programmers 16 and 18.

As already described, the power source 22 housed inside the metallic sleeve 32 provides electrical power to the PCB assembly 34 housed inside the non-conductive housing 26 to power the electronic components 38 supported on its printed circuit board 36. The PCB assembly 34 in turn provides electrical power to the electrodes 74A of a lead 74 extending distally from a strain-relief device 76 that is detachably connected to the feedthrough connector assembly 24 which, in turn, is connected to the proximal weld ring 28 of the non-conductive housing 26.

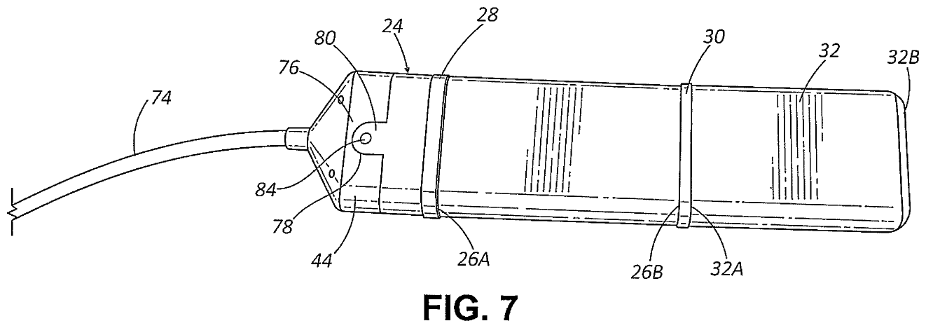

In particular, the strain-relief device 76 has an inlet 76A extending inwardly from an annular sidewall into a beveled sidewall. The inlet 76A has a threaded opening 78. The inlet 76A of the strain-relief device 76 is sized to receive a hub 80 (FIGS. 4 and 7) extending outwardly from the ferrule 44 of the feedthrough 24. The hub 80 also has a threaded opening 82 so that with the feedthrough hub 80 positioned in the inlet 76A of the strain-relief device 76, the respective threaded openings 78, 82 are aligned with each other. A threaded member 84 is then threaded into the aligned openings 78 and 82 to fasten the feedthrough connector assembly 24 to the strain-relief device 76.

The feedthrough connector assembly 24 has a number of terminal pins (not shown), for example, eight which connect to the electrical contacts 48A, 48B, 48C, 48D, 48E, 48F, 48G and 48H supported on the PCB 36 at its proximal end or feedthrough connection end 44. The terminal pins are hermetically sealed in an electrically non-conductive insulator supported in an opening in the ferrule 44 for the feedthrough, as is well known by those skilled in the art of feedthrough assemblies. Suitable feedthrough connector assemblies 24 configured to detachably connect to the strain-relief device 76 for the lead 74 are described in U.S. Pub. Nos. 2023/0372720, 2023/0372721, 2023/0405312, and 2024/0374905, which are assigned to the assignee of the present invention and incorporated herein by reference.

As previously described, the lead 74 extends distally from the strain-relief device 76, which is detachably connected to the feedthrough connector assembly 24 connected to the non-conductive housing 26 for the PCB assembly 34. The lead 74 has a number of electrodes 74A that are configured to deliver current pulses to body tissue, receive sensed electrical signals pertaining to functions of body tissue in which the AMD 12 is implanted or on which the AMD 12 is externally supported, or both sense electrical signals and deliver current pulses.

Thus, the elongated shape of the AMD 12 allows the implantation procedure to be performed by injecting or inserting the AMD into body tissue using of an insertion tool, typically through a very small incision. With the AMD 12 implanted in body tissue, the electrical power source 22 electrically connected to the PCB assembly 34 provides electrical power to the electrodes 74A of the lead 74. The electrodes 74A are configured to electrically stimulate body tissue or sense biological signals, or sense and stimulate.

It is appreciated that various modifications to the inventive concepts described herein may be apparent to those skilled in the art without departing from the spirit and scope of the present invention as defined by the hereinafter appended claims.

What is claimed is:

1. An active medical device (AMD), comprising:
a) a non-conductive housing having an annular sidewall defining an open housing interior;
b) a printed circuit board (PCB) assembly comprising a printed circuit board supporting at least one electronic component, wherein the PCB assembly extends longitudinally to opposed printed circuit board proximal and distal ends; and
d) a communication antenna/inductive charging coil assembly electrically connected to the at least one electronic component of the PCB assembly, wherein the communication antenna/inductive charging coil assembly is also connected to the printed circuit board by a hinge so that in a folded configuration at the hinge, the communication antenna/inductive charging coil assembly is aligned in a spaced relationship with the PCB assembly, and wherein the communication antenna/inductive charging coil assembly folded over the PCB assembly is housed in the open housing interior of the non-conductive housing.

2. The AMD of claim 1, further including:
a) the printed circuit board proximal end supporting a plurality of proximal electrical contacts, and the printed circuit board distal end supporting at least two distal electrical contacts;
b) the non-conductive housing extending from an annular proximal housing rim to an annular distal housing rim;
c) a metallic proximal ring brazed to the proximal housing rim and a metallic distal ring brazed to the distal housing rim;
d) a metallic sleeve extending from a closed distal sleeve end to an annular proximal sleeve rim leading to an open sleeve interior;
e) an electrical power source housed inside the open sleeve interior, wherein the power source has opposite polarity terminals connected to the at least two distal electrical contacts at the distal end of the printed circuit board adjacent to the proximal sleeve rim connected to the distal ring brazed to the distal housing rim of the non-conductive housing; and
f) a feedthrough connector assembly comprising an electrically non-conductive insulator brazed to a ferrule in a ferrule opening, wherein the insulator supports a plurality of hermetically sealed terminal pins comprising terminal pin device side ends extending outwardly beyond an insulator device side and terminal pin body fluid side ends extending outwardly beyond an insulator body fluid side, and wherein the terminal pin device side ends are electrically connected to the plurality of proximal electrical contacts at the proximal end of the printed circuit board adjacent to the proximal housing rim of the non-conductive housing brazed to the proximal ring connected to the ferrule of the feedthrough connector assembly.

3. The AMD of claim 1, wherein the hinge connecting the printed circuit board to the communication antenna/inductive charging coil assembly is a living hinge.

4. The AMD of claim 1, wherein the hinge is a flexible extension from the printed circuit board of the PCB assembly.

5. The AMD of claim 1, wherein the printed circuit board has a first thickness adjacent to the communication antenna/inductive charging coil assembly and the hinge has a second thickness that is less than the first thickness.

6. The AMD of claim 1, wherein the hinge comprises at least two spaced-apart flexible extensions from the printed circuit board.

7. The AMD of claim 2, wherein the feedthrough connector assembly is connectable to a lead.

8. The AMD of claim 7, wherein a proximal end of the lead is connected to a strain-relief device and the strain-relief device is connectable to the feedthrough connector assembly.

9. The AMD of claim 2, wherein the power source is rechargeable.

10. The AMD of claim 9, wherein the communication antenna/inductive charging coil assembly comprises a charging coil connected to a charging circuit on the printed circuit board, and wherein the charging circuit is configured to convert RF or inductive energy received from the charging coil into a direct current voltage to charge the power source to power the PCB assembly.

11. An active medical device (AMD), comprising:
a) a ceramic housing having an annular sidewall defining an open housing interior, wherein the housing extends from an annular proximal housing rim to an annular distal housing rim;
b) a metallic proximal ring brazed to the proximal housing rim and a metallic distal ring brazed to the distal housing rim;
c) a printed circuit board (PCB) assembly comprising a printed circuit board supporting at least one electronic component, the PCB assembly having a length comprising longitudinally extending printed circuit board right and left lateral edges and a width comprising opposed printed circuit board proximal and distal ends, wherein the printed circuit board proximal end supports a plurality of proximal electrical contacts, and the printed circuit board distal end supports at least two distal electrical contacts;
d) a communication antenna/inductive charging coil assembly electrically connected to the at least one electronic component of the PCB assembly, wherein the communication antenna/inductive charging coil assembly is also connected to the printed circuit board by a hinge so that in a folded configuration at the hinge, the communication antenna/inductive charging coil assembly resides spaced above the PCB assembly, and wherein the communication antenna/inductive charging coil assembly folded over the PCB assembly is housed inside the ceramic housing;
e) a metallic sleeve extending from a closed distal sleeve end to an annular proximal sleeve rim leading to an open sleeve interior;
f) an electrical power source housed inside the open sleeve interior, wherein the power source has opposite polarity terminals connected to the at least two distal electrical contacts at the distal end of the printed circuit board adjacent to the proximal sleeve rim connected to the distal ring brazed to the distal housing rim of the ceramic housing; and
g) a feedthrough connector assembly comprising an electrically non-conductive insulator brazed to a ferrule in a ferrule opening, wherein the insulator supports a plurality of hermetically sealed terminal pins comprising terminal pin device side ends extending outwardly beyond an insulator device side and terminal pin body fluid side ends extending outwardly beyond an insulator body fluid side, and wherein the terminal pin device side ends are electrically connected to the plurality of proximal electrical contacts at the proximal end of the printed circuit board adjacent to the proximal housing rim of the ceramic housing brazed to the proximal ring connected to the ferrule of the feedthrough connector assembly.

12. The AMD of claim 11, wherein the hinge is connected to one of the printed circuit board right lateral edge, left lateral edge, proximal end, and distal end.

13. The AMD of claim 11, wherein the hinge is a flexible extension from the printed circuit board of the PCB assembly.

14. The AMD of claim 11, wherein the printed circuit board has a first thickness adjacent to the communication antenna/inductive charging coil assembly and the hinge has a second thickness that is less than the first thickness.

15. The AMD of claim 11, wherein the hinge comprises at least two spaced-apart flexible extensions from the printed circuit board.

16. The AMD of claim 11, wherein the feedthrough connector assembly is connectable to a lead.

17. The AMD of claim 16, wherein a proximal end of the lead is connected to a strain-relief device and the strain-relief device is connectable to the feedthrough connector assembly.

18. The AMD of claim 10, wherein the power source is rechargeable.

19. The AMD of claim 18, wherein the communication antenna/inductive charging coil assembly comprises a charging coil connected to a charging circuit on the printed circuit board, and wherein the charging circuit is configured to convert RF or inductive energy received from the charging coil into a direct current voltage to charge the power source to power the PCB assembly.

20. The AMD of claim 11, wherein the communication antenna/inductive charging coil assembly has a shaped selected from a rectangular shape, an oval shape, a circular shape, a multi-sided shape that is neither rectangular or circular but has both straight sides and curved sides, and wherein the communication antenna/inductive charging coil assembly either has or does not have a length and a width that substantially aligns with the length and the width of the PCB assembly.
